(19) United States
(12) Patent Application Publication
(10) Pub. No.: US 2004/0260394 A1
(43) Pub. Date: Dec. 23, 2004

(54) Cardiac valve annulus compressor system

(75) Inventors: Nareak Douk, Lowell, MA (US); Nasser Rafiee, Andover, MA (US); Eliot Bloom, Hopkinton, NH (US)

Correspondence Address: Catherine C. Maresh, IP Legal, 3576 Unocal Place, Santa Rosa, CA, 95403, US

(73) Assignee: MEDTRONIC VASCULAR, INC.

(21) Appl. No.: 10/867,471

(22) Filed: Jun. 14, 2004

Related U.S. Application Data
(60) Provisional application No. 60/480,062, filed on Jun. 20, 2003.

(51) Int. Cl. A61F 2/24

(52) U.S. Cl. 623/2.36; 606/195; 606/153

(57) ABSTRACT

A cardiac valve annulus compressor comprises a generally cylindrical main body having plain and barbed ends and an actuator portion. Barbs disposed on the barbed end are engageable with the valve annulus. The length of the circumference of the barbed end is responsive to movement of the actuator portion. The annulus compressor can be delivered percutaneously or surgically. A cardiac valve annulus compressor system and methods of use are also taught.

### PRIORITY CLAIM

This application claims the benefit of U.S. Provisional Application No. 60/480,062 filed Jun. 20, 2003; the entirety of each of which is hereby incorporated by reference.

### TECHNICAL FIELD

The technical field of this disclosure is medical devices, particularly, a cardiac valve annulus compressor system and method of using the same.

### BACKGROUND OF THE INVENTION

Heart valves, such as the mitral, tricuspid, aortic, and pulmonic valves, are sometimes damaged by diseases or by aging, which can cause problems with the proper function of the valve. Heart valve disease generally takes one of two forms: stenosis, in which a valve does not open completely or the opening is too small, resulting in restricted blood flow; or insufficiency, in which blood leaks retrograde across the valve that should be closed. Valve repair or replacement may be required in severe cases to restore cardiac function. In common practice, repair or replacement requires open-heart surgery with its attendant risks, expense, and extended recovery time. Open-heart surgery also requires cardiopulmonary bypass with risk of thrombosis, stroke, and infarction.

Catheter based valve repair using mechanical devices to remodel the cardiac valve has been proposed as a way to effect valve repair percutaneously and avoid open-heart surgery. Such repair systems typically lack the capacity for fine adjustment during or after remodeling or repair. Fine adjustment during valve remodeling or repair is desirable to assure that the remodeling results in proper valve function. Valve size and shape vary with a particular patient's cardiac problem and structure, so a one-size-fits-all approach can produce less than optimal results. Fine adjustment after valve repair or remodeling is desirable to correct any remaining or newly developed valve problems without open-heart surgery.

U.S. patent application No. 20020099439 to Schwartz et al. discloses a device and method for replacing or restoring competence to incompetent valves. The device generally comprises a venuloplasty ring, which contracts the size of a targeted vein near a native valve that has been rendered incompetent due to venular dilation.

It would be desirable to have a cardiac valve annulus compressor system and method of using the same that would overcome the above disadvantages.

### SUMMARY OF THE INVENTION

One aspect of the present invention provides a cardiac valve annulus compressor system. The cardiac valve annulus compressor system comprises a catheter having a lumen, at least one balloon operably attached to a distal end of the catheter and in fluid communication with the lumen and an annulus compressor disposed about the at least one balloon.

Another aspect of the present invention provides an annulus compressor for compressing a valve annulus. The annulus compressor comprises a generally cylindrical main body having a plain end and a barbed end, an actuator portion operably attached to the main body and barbs disposed on the barbed end, the barbs are engageable with the valve annulus. Further, the length of the circumference of the barbed end is responsive to movement of the actuator portion.

Another aspect of the present invention provides a method for compressing a valve annulus. The method comprises providing an annulus compressor having a barbed end and an actuator portion, implanting the barbed end at the valve annulus and moving the actuator portion to reduce the circumference of the barbed end.

Another aspect of the present invention provides an annulus compressor system. The annulus compressor system comprises means for compressing a valve annulus having a barbed end, means for implanting the barbed end at the valve annulus and means for reducing the circumference of the barbed end.

Another aspect of the present invention provides a cardiac valve annulus compressor system to provide post implantation adjustment without open-heart surgery.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof. The accompanying drawings are not to scale.

### BRIEF DESCRIPTION OF THE DRAWINGS

FIG. 1 shows a cardiac valve annulus compressor and delivery system made in accordance with the present invention.

FIG. 2 shows a cardiac valve annulus compressor delivery system made in accordance with the present invention deploying an annulus compressor.

FIG. 3 shows a cardiac valve annulus compressor made in accordance with the present invention.

FIGS. 4-7 show deployment of a cardiac valve annulus compressor made in accordance with the present invention.

FIGS. 8-9 show a perspective and detail view, respectively, of another cardiac valve annulus compressor made in accordance with the present invention.

FIGS. 10-13 show deployment of another cardiac valve annulus compressor made in accordance with the present invention.

FIG. 14 shows a flow chart for a method of use for a cardiac valve annulus compressor made in accordance with the present invention.

### DETAILED DESCRIPTION OF THE INVENTION

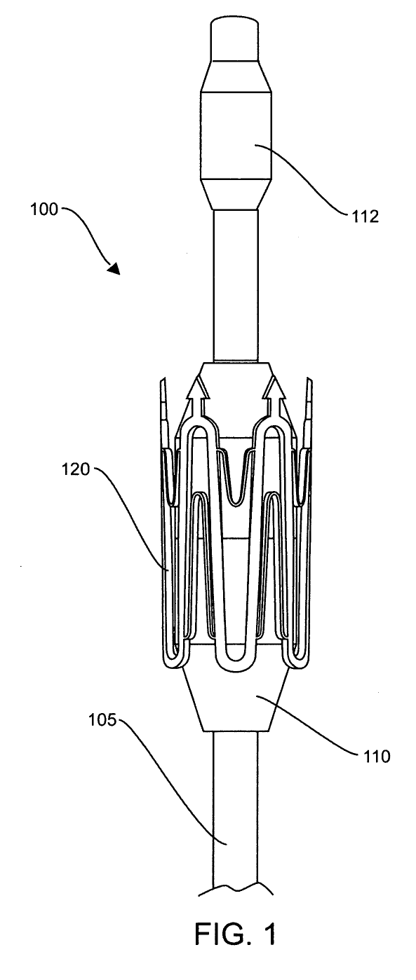

FIG. 1 shows a cardiac valve annulus compressor and delivery system made in accordance with the present invention. The annulus compressor and delivery system 100 includes a catheter 105, a balloon 110 operably attached to the catheter 105, and an annulus compressor 120 disposed on the balloon 110. The balloon 110, shown in a collapsed state, may be any variety of balloons capable of expanding the annulus compressor 120. The balloon 110 may comprise a proximal balloon and a distal balloon to expand different portions of the annulus compressor 120. The balloon 110 may be manufactured from a material such as polyethylene, polyethylene terephthalate (PET), nylon, polyether-block co-polyamide polymer such as PEBAX® resin by AtoFina Chemicals, Inc., or the like. In one embodiment, the balloon is expanded by pressurized fluid. The fluid may be, for example, saline, radiopaque dye, contrast medium, gas or any other suitable fluid for expanding the balloon. In one embodiment, the balloon 110 may include mechanical or adhesive structures for retaining the annulus compressor 120 until it is deployed. The catheter 105 may be any variety of balloon catheters, such as a PTA (percutaneous transluminal angioplasty) balloon catheter, capable of supporting a balloon during angioplasty. The annulus compressor and delivery system 100 may also include an optional guide balloon 112 which can be inflated in the cardiac valve to guide the annulus compressor 120 into position for deployment.

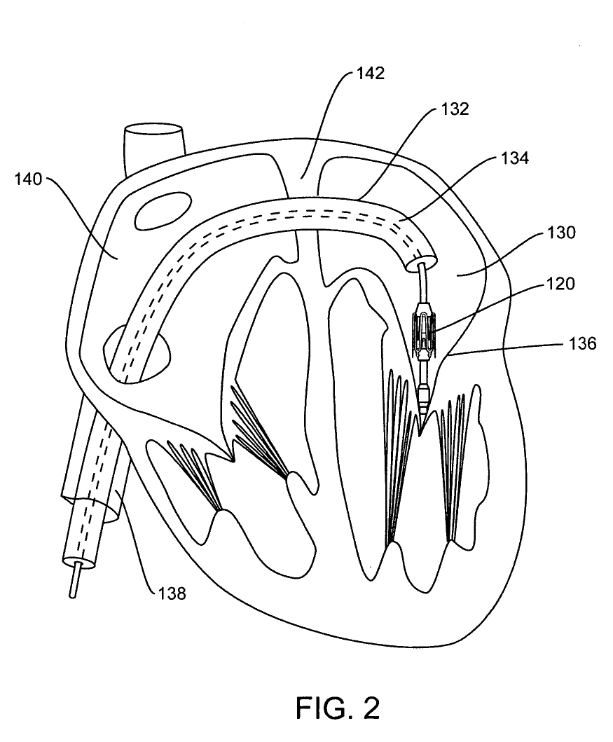

FIG. 2 shows a cardiac valve annulus compressor delivery system made in accordance with the present invention deploying an annulus compressor. The annulus compressor can be delivered percutaneously, using a catheter or mechanical means to expand the annulus compressor. Alternatively, the annulus compressor can be delivered surgically.

For the exemplary case of mitral valve remodeling shown in FIG. 2, the annulus compressor 120 is implanted from the left atrium 130. An elongate element 132 having a lumen 134, such as a catheter, is first installed to provide a path for the annulus compressor delivery system from the exterior of the patient to the left atrium 130. The annulus compressor delivery system can then be advanced through the lumen 134 so that the annulus compressor 120 is located at the mitral valve annulus 136 for deployment. FIG. 2 illustrates a transeptal approach through the vena cava: the elongate element 132 is inserted through the femoral vein into the common iliac vein, through the inferior vena cava 138 into the right atrium 140. The transeptal wall 142 between the right atrium 140 and left atrium 130 is then punctured with a guide wire or other puncturing device, and the distal end of the elongate element 132 advanced into the left atrium 130. The annulus compressor 120 can then be advanced through the lumen 134 of the elongate element 132 to the mitral valve for implantation.

Those skilled in the art will appreciate that alternative paths to gain access to the left atrium are available. For example, another possible path would be through the radial vein into the brachial vein, through the subclavian vein, through the superior vena cava into the right atrium, and then transeptally into the left atrium. Yet another possible path would be through the femoral artery into the aorta, through the aortic valve into the left ventricle, and then retrograde through the mitral valve into the left atrium. For surgical approaches with an open heart, the elongate element can be a trocar or cannula inserted directly in the superior vena cava or the aortic arch. The elongate element can then follow the same path as the percutaneous procedure to reach the left atrium, either transeptally or through the cardiac valves. Transeptal approaches, whether percutaneous or surgical, may require placement of a closure device at the transeptal puncture on removal of the elongate element after the procedure. Similar percutaneous or surgical approaches can be used to access the other cardiac valves, if the annular compressor is to be implanted on a cardiac valve other than the mitral valve.

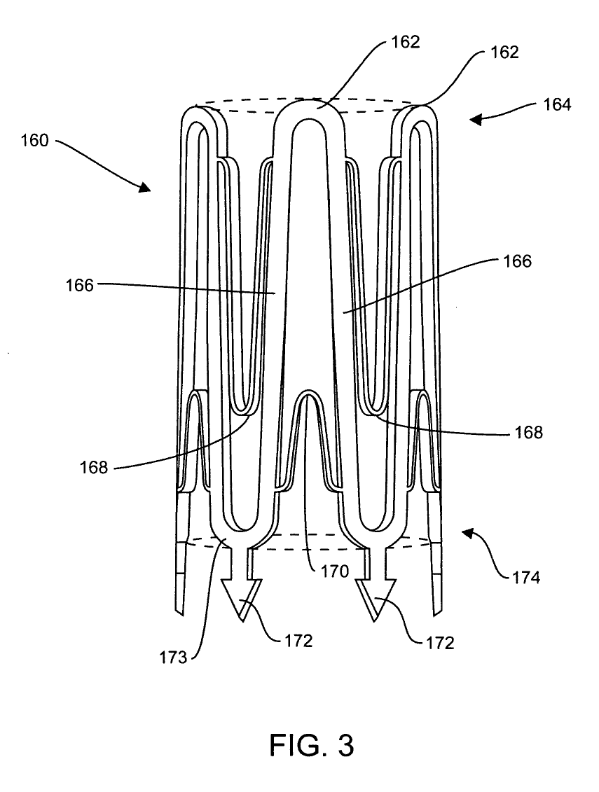

FIG. 3 shows a cardiac valve annulus compressor made in accordance with the present invention. The annulus compressor 160 is shown in the compressed configuration. The annulus compressor 160 comprises a plurality of plain apexes 162 and barbed apexes 173 joined by main struts 166. The main struts 166 define the main body of the annulus compressor 160. Those skilled in the art will appreciate that patterns for the main struts 166 can be zig-zag, interlocking, or any other pattern allowing the annulus compressor 160 to expand radially.

Barbs 172 are disposed on the barbed apexes 173. The barbs 172 can be directed axially parallel to the central axis of the annulus compressor 160, or can be directed radially inward or outward, as required for a particular application. The plain apexes 162 form a plain end 164 and the barbed apexes 173 form a barbed end 174 of the annulus compressor 160, which is generally cylindrical in the compressed configuration. The plain end 164 is the actuator portion of the annulus compressor 160. Pivot struts 170 join the main struts 166 near the barbed end 174. The pivot struts 170 provide a ring for adjustment of the circumference of the barbed end 174 relative to the plain end 164 when the annulus compressor 160 is in the expanded configuration and the valve annulus is being compressed. Essentially, pivot struts 170 act as a first class lever array around the generally cylindrical body of the annulus compressor 160. The arrangement of the pivot struts 170 allows the compression of the valve annulus when the embedded annulus compressor 160 is radially expanded at the plain end 164 by the proximal balloon 192. Limit struts 168 join the main struts 166 near the plain end 164. In one embodiment, fully expanded limit struts act as a locking mechanism to lock the fully expanded annulus compressor into the desired configuration. The length and position of the pivot struts 170 and the limit struts 168 can be varied to produce the desired geometry for the deployed annulus compressor 160 and to produce the desired action during adjustment. In other embodiments, the limit struts can be omitted.

The annulus compressor 160 can be made of any biocompatible material, which can be compressed for delivery to the cardiac valve and expanded mechanically or self-expanded to compress the valve annulus. In one embodiment, the annulus compressor 160 can be made of stainless steel or a cobalt-based metal like MP35N® alloy by SPS Technologies, Inc. In another embodiment, the annulus compressor 160 can be made of a memory metal, such as nitinol. The annulus compressor 160 can be fabricated by laser or mechanical cutting methods well known in the art.

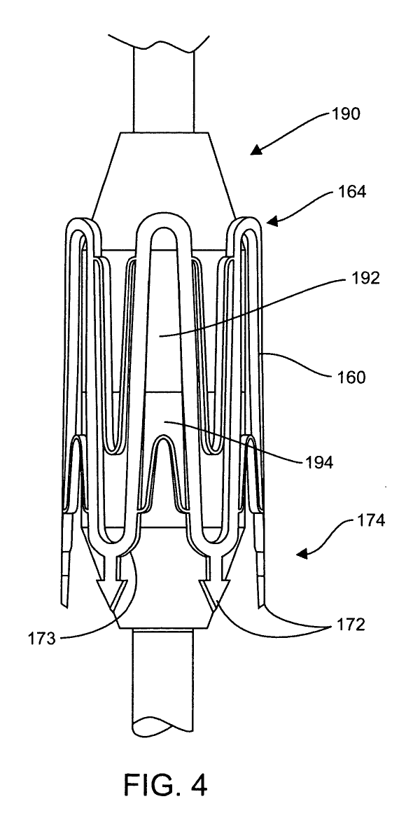

FIGS. 4-7, in which like elements share like reference numbers with FIG. 3, show deployment of a cardiac valve annulus compressor made in accordance with the present invention. The sequence of figures shows, respectively, the annulus compressor in the compressed configuration on a balloon, expanded with the distal balloon, adjusted with the proximal balloon to compress the annulus, and in the deployed configuration.

Referring to FIG. 4, the annulus compressor 160 is disposed on a balloon catheter 190, which comprises a proximal balloon 192 and a distal balloon 194. The plain end 164 of the annulus compressor 160 is disposed about the proximal balloon 192 and the barbed end 174 of the annulus compressor 160 is disposed about the distal balloon 194. The barbs extend axially from the barb apexes 173. The balloons expand with fluid pressure provided through lumens (not shown) in the catheter. The proximal balloon 192 and the distal balloon 194 can be expanded independently of each other. The compressed configuration shown in FIG. 4 is used to introduce the annulus compressor 160 into the body and to the implantation site at the cardiac valve annulus via a delivery catheter 190.

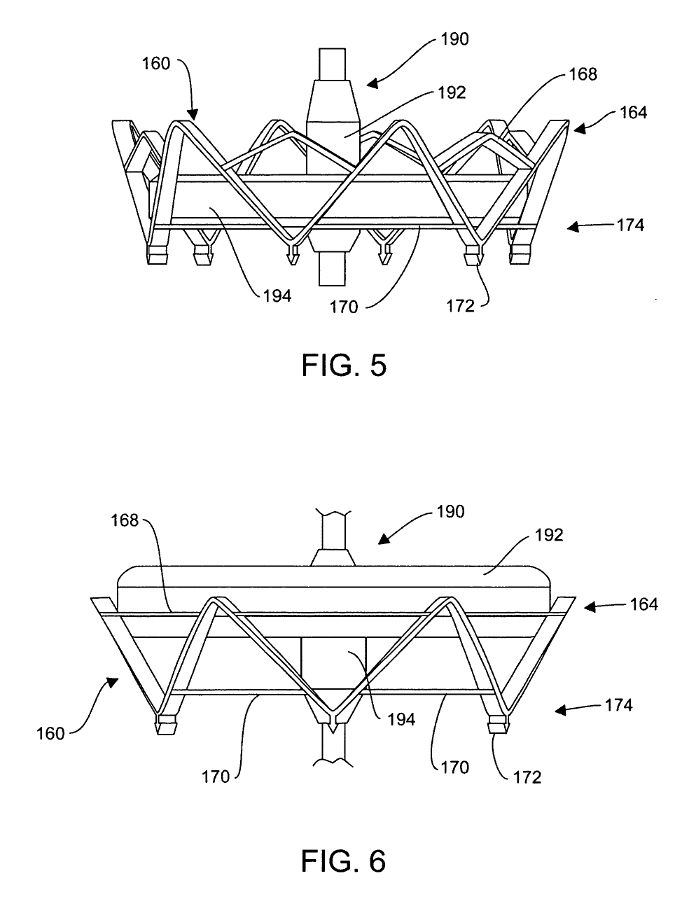

Referring to FIG. 5, once the annulus compressor 160 is delivered adjacent the cardiac valve annulus, the distal balloon 194 is inflated to expand the annulus compressor 160 radially and to place barbs 172 in contact with or adjacent to the valve annulus. The pivot struts 170 can be fully expanded. The barbs 172 are seated in the valve annulus by applying axial pressure along the axis of the catheter.

Referring to FIG. 6, the distal balloon 194 is deflated and the proximal balloon 192 is inflated to compress the valve annulus. As the proximal balloon 192 inflates, the circumference of the plain end 164 expands to the length of the limit struts 168 and the circumference of the barbed end 174 is reduced. The barbs 172 apply pressure to the valve annulus to remodel the cardiac valve. The effectiveness of the compression in remodeling the cardiac valve can be monitored as the proximal balloon 192 expands. The remodeling may be monitored by fluoroscopy, ultrasonography or any other method known to those with skill in the art. Where fluoroscopy is utilized, the delivery catheter 190, balloon catheter and/or annulus compressor may contain radiopaque markers or may be composed of radiopaque material for viewing under fluoroscopy as is known in the art. Where ultrasonography is utilized, the delivery catheter 190, balloon catheter and/or annulus compressor may contain a coating or may be composed of a material having a density substantially different than the surrounding tissue as is known in the art.

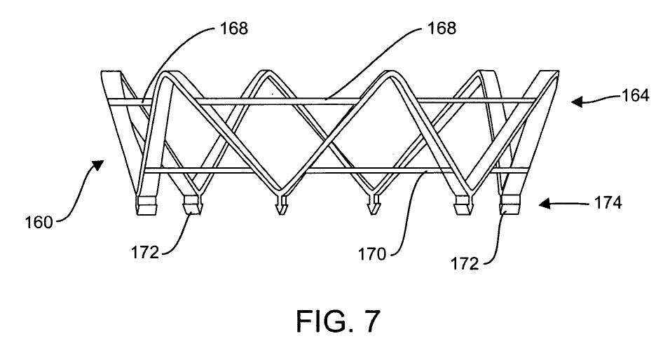

Referring to FIG. 7, the proximal balloon is deflated and the catheter removed, leaving the annulus compressor 160 in the final deployed configuration. The annulus compressor 160 maintains compression on the valve annulus to correct valve function.

Those skilled in the art will appreciate that the procedure presented in FIGS. 4-7 can be varied to equal effect. For example, a single chamber balloon can be used and the catheter moved axially relative to the annulus compressor to expand the particular portions of the annulus compressor.

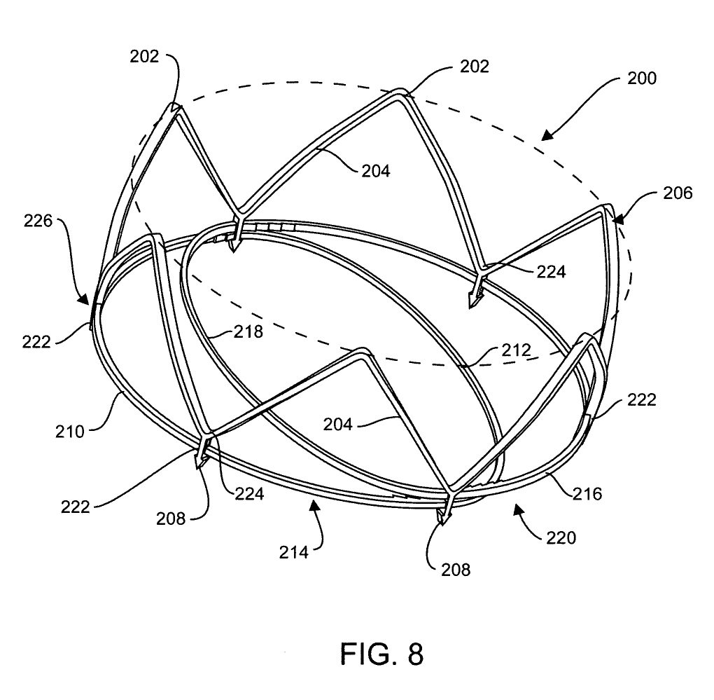

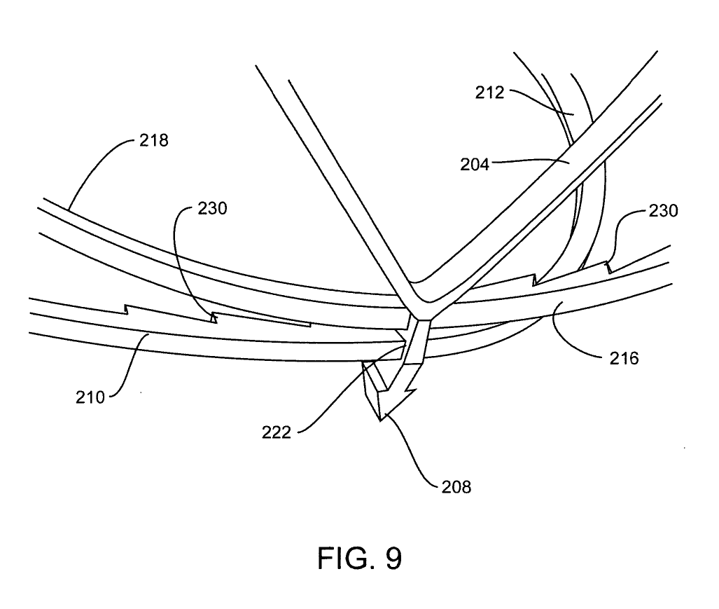

FIGS. 8-9 show a perspective and detail view, respectively, of another cardiac valve annulus compressor made in accordance with the present invention. In FIG. 8 the annulus compressor 200 is shown in the partially expanded configuration. The annulus compressor 200 comprises a plurality of plain apexes 202 and barbed apexes 224 joined by main struts 204. The main struts 204 define the generally cylindrical main body of the annulus compressor 200. Those skilled in the art will appreciate that patterns for the main struts 204 can be zigzag, interlocking, or any other pattern allowing the annulus compressor 200 to expand radially.

Barbs 208 are disposed on the barbed apexes 224 and include an opening 222 through which the cords 214, 220 can pass. The barbs 208 can be directed axially in line with the central axis of the annulus compressor 200, or can be directed radially inward or outward, as required for a particular application. The plain apexes 202 form a plain end 206 and the barbed apexes 224 form a barbed end 226 of the annulus compressor 200, which is generally cylindrical in the compressed configuration. In another embodiment, limit struts, similar to those limit struts 168 shown in the previous embodiment, can be attached to the main struts 204 near the plain end 206.

The circumference of the barbed end 226 can be adjusted with cords to compress the valve annulus. The cord can be any metal bar or wire of sufficient flexibility to bend around the outer circumference of the barbed end 226. First cord 214 comprises a ring portion 210 and an interlocking portion 212. Second cord 220 comprises a ring portion 216 and an interlocking portion 218. The ring portions 210, 216 of each cord 214, 220 pass through the openings 222 in the barbs 208. In essence, the arrangement of the cords 214, 220 in the overlapping configuration is similar in appearance to a Venn diagram. The overlapping space between interlocking portions 212, 218 defines the actuator portion of the annulus compressor 200. Expanding a balloon in the overlapping space between interlocking portions 212, 218 forces the interlocking portions 212, 218 apart, sliding the ring portions 210, 216 in the openings 222 to reduce the circumference of the barbed end 226. A locking mechanism, described below, maintains the reduced circumference.

Referring to FIG. 9, ratchet teeth 230 on the ring portions 210, 216 lock the circumference of the barbed end 226. Once a ratchet tooth 230 engages the opening 222, the ring portions 210, 216 cannot slide backwards through the opening 222. In other embodiments, each ring portion 210, 216 can pass through its own opening. In another embodiment, the ratchet teeth can be disposed within the opening 222, rather than on the ring portions 210, 216. In this embodiment, each ring would include a series of projections to engage the ratchet teeth. Any mechanism allowing cord motion in one direction and preventing cord motion in the opposite direction is suitable for the locking mechanism.

The annulus compressor 200 can be made of any biocompatible material, which can be compressed for delivery to the cardiac valve and expanded mechanically or self-expanded to compress the valve annulus. In one embodiment, the annulus compressor 200 can be made of stainless steel or cobalt-based metal alloy such as, for example, MP35N. In another embodiment, the annulus compressor 200 can be made of a memory metal, such as nitinol. The annulus compressor 200 can be fabricated by laser or mechanical cutting methods well known in the art.

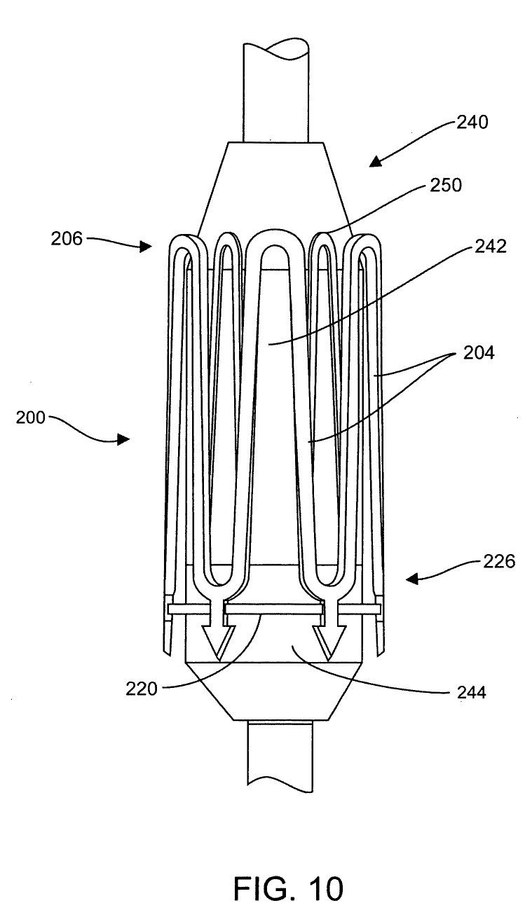

FIGS. 10-13, in which like elements share like reference numbers with FIG. 9, show deployment of another cardiac valve annulus compressor made in accordance with the present invention. The sequence of figures shows, respectively, the annulus compressor in the compressed configuration on a balloon, after expansion with the proximal balloon, axially during adjustment to compress the annulus with the distal balloon, and in the deployed configuration.

Referring to FIG. 10, the annulus compressor 200 is disposed on a balloon catheter 240, which comprises a proximal balloon 242 and a distal balloon 244. The plain end 206 of the annulus compressor 200 is disposed about the proximal balloon 242 and the barbed end 226 of the annulus compressor 200 with the cord 220 is disposed about the distal balloon 244. The balloons expand with fluid pressure provided through lumens (not shown) in the catheter 240. The proximal balloon 242 and the distal balloon 244 can be expanded independently of each other. Limit struts 250 are attached to the main struts 204. The compressed configuration shown in FIG. 10 is used to introduce the annulus compressor 200 into the body and to the implantation site at the cardiac valve annulus.

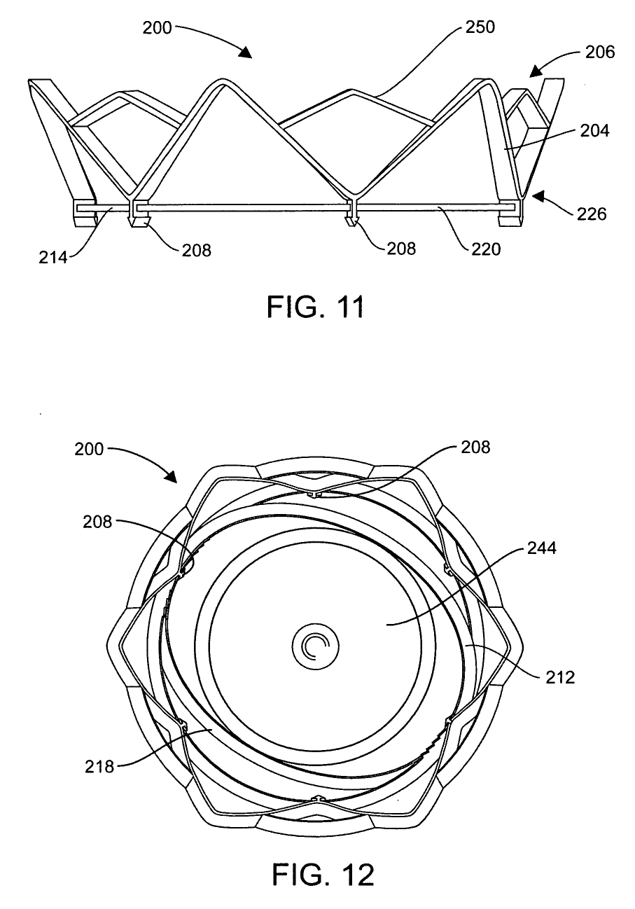

FIG. 11 shows the annulus compressor 200 after expansion with the proximal balloon (not shown) to seat the barbs 208 in the valve annulus. The radial expansion of the proximal balloon and axial pressure along the axis of the catheter seats the barbs 208 in the valve annulus. The limit struts 250 have been partially expanded by inflation of the proximal balloon 242.

Referring to FIG. 12, the distal balloon 244 is inflated to compress the valve annulus. The expansion of the distal balloon 244 spreads the interlocking portions 212, 218 apart to reduce the circumference of the barbed end. The barbs 208 apply inward radial forces to the valve annulus to remodel the cardiac valve. The effectiveness of the compression in remodeling the cardiac valve can be monitored as the distal balloon 244 expands. The circumference of the barbed end is locked into position when the remodeling is satisfactory using the locking mechanism described above.

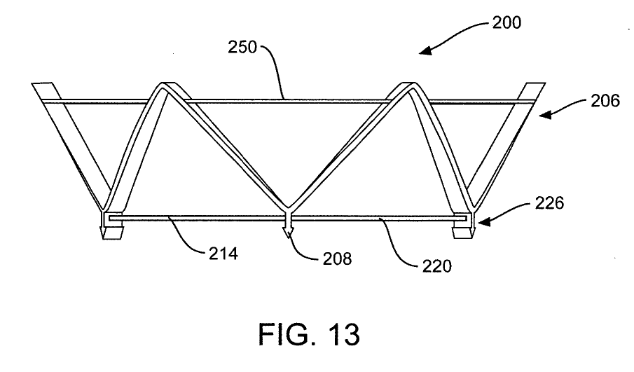

Referring to FIG. 13, the distal balloon has been deflated and the catheter removed, leaving the annulus compressor 200 in the final deployed configuration. The annulus compressor 200 maintains compression on the valve annulus to correct valve function. In one embodiment, the limit struts 250 are fully extended.

Those skilled in the art will appreciate that the procedure presented in FIGS. 10-13 can be varied to equal effect. For example, a single chamber balloon can be used and the catheter moved axially relative to the annulus compressor to expand the particular portions of the annulus compressor.

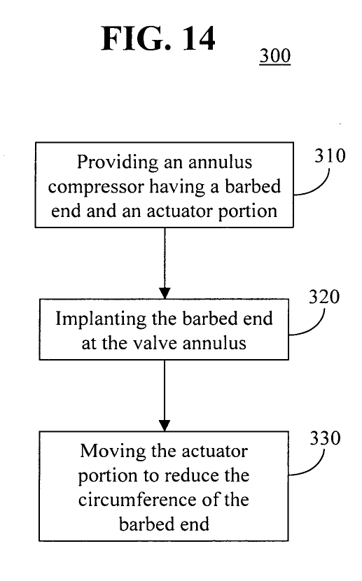

FIG. 14 shows a flow chart for a method of using a cardiac valve annulus compressor made in accordance with the present invention and generally referred to as method 300. The method begins by providing an annulus compressor having a barbed end and an actuator portion (Block 310). As described above, the annulus compressor is advanced to the treatment site adjacent to the valve annulus via a catheter inserted percutaneously. In another embodiment, the annulus compressor can be delivered surgically. Method 300 continues by implanting the barbed end at the valve annulus (Block 320). In one embodiment, the barbs are implanted by first radially expanding the barbed end of the annulus compressor and then applying axial pressure to the catheter to embed the barbs. Method 300 continues by moving the actuator portion of the annulus compressor to reduce the circumference of the barbed end (Block 330). In one embodiment, the annulus compressor has a plain end, which is expanded to reduce the circumference of the barbed end. In another embodiment, the annulus compressor has a first cord having a first interlocking portion and a second cord comprising a second interlocking portion. The first cord and the second cord are slidably disposed in the barbed end, so that expanding the area between the first interlocking portion and the second interlocking portion reduces the circumference of the barbed end.

In another embodiment, the method 300 can further comprise locating the annulus compressor at the valve annulus using a guide balloon or other locating device. In yet another embodiment, the method can further comprise monitoring remodeling effectiveness while moving the actuator portion to reduce the circumference of the barbed end.

It is important to note that FIGS. 1-14 illustrate specific applications and embodiments of the present invention, and is not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A cardiac valve annulus compressor system comprising:
a catheter having a lumen;
at least one balloon operably attached to a distal end of the catheter, the balloon being in fluid communication with the lumen; and
an annulus compressor disposed about the at least one balloon.

2. The annulus compressor system of claim 1, wherein:
the at least one balloon comprises a proximal balloon and a distal balloon;
the annulus compressor includes a plain end and a barbed end;
the plain end is disposed on the proximal balloon; and
the barbed end is disposed on the distal balloon.

3. The annulus compressor system of claim 1, further comprising a guide balloon disposed on the catheter.

4. The annulus compressor system of claim 1, wherein the annulus compressor comprises:
a main body, the main body including a plain end and a barbed end;
an actuator portion, the actuator portion operably attached to the main body; and
barbs disposed on the barbed end, the barbs being engageable with a valve annulus;
wherein the length of the circumference of the barbed end is responsive to movement of the actuator portion.

5. An annulus compressor for compressing a valve annulus comprising:
a generally cylindrical main body, the main body having a plain end and a barbed end;
an actuator portion, the actuator portion operably attached to the main body; and
barbs disposed on the barbed end, the barbs being engageable with the valve annulus;
wherein the length of the circumference of the barbed end is responsive to movement of the actuator portion.

6. The annulus compressor of claim 5, wherein the circumference of the barbed end is smaller than the circumference of the plain end when the annulus compressor is in a deployed configuration.

7. The annulus compressor of claim 5, wherein:
the plain end comprises a plurality of plain apexes;
the barbed end comprises a plurality of barbed apexes;
main struts connect the plurality of plain apexes and the plurality of barbed apexes;
pivot struts connect the main struts near the barbed end; and
the actuator portion comprises the plain end.

8. The annulus compressor of claim 7, further comprising limit struts connecting the main struts near the plain end.

9. The annulus compressor of claim 7, wherein the direction of the barbs relative to a central axis of the annulus compressor is selected from the group consisting of parallel to the central axis, radially inward toward the central axis, and radially outward away from the central axis.

10. The annulus compressor of claim 5, wherein:
the plain end comprises a plurality of plain apexes;
the barbed end comprises a plurality of barbed apexes;
main struts connect the plurality of plain apexes and the plurality of barbed apexes; and further comprising:
a first cord, the first cord comprising a first ring portion and a first interlocking portion;
a second cord comprising a second ring portion and a second interlocking portion;
the first ring portion and the second ring portion pass through openings in the barbs; and
the actuator portion comprises the first interlocking portion and the second interlocking portion.

11. The annulus compressor of claim 10, wherein the first ring portion and the second ring portion have ratchet teeth to engage the opening, so that the circumference of the barbed end cannot expand.

12. The annulus compressor of claim 10, further comprising limit struts connecting the main struts near the plain end.

13. An annulus compressor system comprising:
means for compressing a valve annulus having a barbed end;
means for implanting the barbed end at the valve annulus; and
means for reducing the circumference of the barbed end.

14. The system of claim 13 further comprising means for delivering the annulus compressor to the valve annulus.

15. The system of claim 13 wherein:
the valve annulus compressing means has a plain end; and
the means to reduce the circumference of the barbed end comprises expanding the plain end.

16. The system of claim 13 wherein:
the valve annulus compressing means has a first cord having a first interlocking portion and a second cord having a second interlocking portion; and
the first cord and the second cord are slidably disposed in the barbed end.

17. The system of claim 16 further comprising means for locking the first cord and the second cord in position.

18. The system of claim 13 further comprising means for locating the annulus compressor at the valve annulus.

19. The system of claim 13 further comprising means for monitoring remodeling effectiveness.

20. A method for compressing a valve annulus comprising:
providing an annulus compressor having a barbed end and an actuator portion;
implanting the barbed end at the valve annulus; and
moving the actuator portion to reduce the circumference of the barbed end.

21. The method of claim 20 further comprising delivering the annulus compressor to the valve annulus by the method selected from the group consisting of percutaneous delivery and surgical delivery.

22. The method of claim 20 wherein:
the annulus compressor has a plain end; and
moving the actuator portion to reduce the circumference of the barbed end comprises expanding the plain end.

23. The method of claim 20 wherein:
the annulus compressor has a first cord having a first interlocking portion and a second cord comprising a second interlocking portion;
the first cord and the second cord are slidably disposed in the barbed end; and
moving the actuator portion to reduce the circumference of the barbed end comprises expanding an area between the first interlocking portion and the second interlocking portion.

24. The method of claim 20 further comprising locating the annulus compressor at the valve annulus.

25. The method of claim 24 wherein locating the annulus compressor at the valve annulus comprises locating the annulus compressor at the valve annulus using a guide balloon.

26. The method of claim 20 further comprising monitoring remodeling effectiveness while moving the actuator portion to reduce the circumference of the barbed end.
